(19) United States
(12) Patent Application Publication
(10) Pub. No.: US 2024/0091333 A1
(43) Pub. Date: Mar. 21, 2024

(54) Temperature Stable Nucleic Acid Method for Preparing Vaccines

(71) Applicant: Douglas T. Gjerde, Saratoga, CA (US)

(72) Inventor: Douglas T. Gjerde, Saratoga, CA (US)

(21) Appl. No.: 18/269,925

(22) PCT Filed: Dec. 28, 2021
(86) PCT No.: PCT/US21/65395

§ 371 Date: Jun. 27, 2023

Related U.S. Application Data
(60) Provisional application No. 63/131,258, filed on Dec. 28, 2020.
(60) Provisional application No. 63/239,904, filed on Sep. 1, 2021.
(60) Provisional application No. 63/241,083, filed on Sep. 6, 2021.

(51) Int. Cl. A61K 39/085; A61K 9/19

(52) U.S. Cl. CPC A61K 39/085; A61K 9/19; A61K 2039/55555

(57) ABSTRACT

Nucleic acid and the nanocomplex reagents are combined to create a vaccine. They are stable and stored separately without degradation. The vaccine components can be stored at a wide range of temperatures. The nucleic acids are stabilized and stored in a column, syringe, vial or chamber as a solid, lyophilized or precipitated. They may be stored on a solid phase surface through electrostatic forces, non-polar interactions, hydrogen bonding, polar interactions or any other mechanism. The solid surface may be media in a column which may be contained in a syringe. Nucleic acid vaccines are prepared by a two-step process. The nucleic acid component is first stabilized and then mixed with nanocomplex reagents, particle forming reagents or other reagents.

### BACKGROUND OF THE INVENTION

Vaccination and immunization activate the immune system against a specific antigen or group of antigens. Vaccination can be performed in different ways including delivery of a killed or weakened whole pathogen, delivery of a recombinant protein or peptide mimicking a natural antigen/epitope or delivery of a nucleic acid to the cell encoding a specific antigen.

Plasmid DNA, mRNA, and siRNA are examples of nucleic acids modulating gene expression. The principle of nucleic acid vaccination is to inject into humans and transfect in the cells a genetically engineered plasmid, RNA or nucleic acid encoding a pathogen-specific antigen. The transfected cells express/produce the desired antigen into the extracellular space resulting in the stimulation of a protective immune response. Examples include immunization against human papillomavirus, hepatitis B virus, and respiratory pathogens such as vaccinia virus and influenza virus. One of the successful strategies for the development of vaccines against COVID-19 is the transfection of mRNA into cells. In many cases, nucleic acid vaccines have been developed to protect against viral pathogens. Recent nucleic acid strategies have been used to promote antibodies against tumor-associated antigens in cancer cells. Nucleic acid vaccines can be directed against any cell or antigen. The nucleic acid in the vaccine will use the machinery of the cell to produce proteins. The vaccine is a substance used to stimulate the production of antibodies against disease. An immune response is directed against these proteins to combat various diseases including viral infection, cancer and other diseases.

Both DNA and mRNA vaccines have been under development for several decades. Plasmid DNA vaccines must enter the cell nucleus, unlike mRNA vaccines, which needs only to enter the cytoplasm. While RNA may be injected with a syringe and needle, plasmid DNA may be deposited under the skin, as opposed to deep in muscle tissue. The area beneath the skin is rich in immune cells that will interact with the vaccine particles and process them. DNA may be captured more easily and, in some cases, the vaccine is delivered with a patch or device pressed against the skin. This creates a fine, high-pressure stream of fluid that punctures the surface.

Different delivery methods exist to introduce nucleic acids from vaccines into tissue cells. These include the direct injection of a saline solution of RNA in the muscles. Naked mRNA, at least to a small degree, can cross the cell lipid bilayer to reach cytoplasm. Other physical delivery methods across the lipid bilayer include the gene gun or in vivo electroporation.

However, these are not efficient methods. Nucleic acids are negatively charged and the cell wall lipid bilayer is negatively charged. Transfection of the nucleic acid into the cell requires overcoming the repulsion of the exposed negative charges of the lipid bilayer. A nucleic acid carrier envelope is usually required to shield the negative charges and allow the nucleic acid to travel through the cell wall more easily. Delivery of the nucleic acid with the aid of transfection reagents is quite useful and successful. Synthetic nucleic acid delivery agents include liposomes and polymers that carry cationic charges. These reagents aid formation of the nucleic acid into nanoparticle complexes. RNA and DNA may be enveloped with different chemicals including linear polyethylenimine-based reagents or lipid-based reagents and many others. The complexes are sometimes called lipid nanocomplexes or cationic liposomes. Other reagents, some of them proprietary, can be used as transfection reagents.

For example, lipids are employed in the Pfizer COVID vaccine. Their main role is to protect the mRNA and provide somewhat of a “greasy” exterior that helps the mRNA slide inside cells. Examples include ((4-hydroxybutyl)azanediyl)bis(hexane-6,1-diyl)bis, (2-hexyldecanoate), 2 [(polyethylene glycol)-2000]-N,N-ditetradecylacetamide, and 1,2-Distearoyl-snglycero-3-phosphocholine and cholesterol. The following salts are also included in the Pfizer vaccine and help balance the acidity in the body: potassium chloride, monobasic potassium phosphate, sodium chloride and dibasic sodium phosphate dihydrate. Sucrose, a sugar is also used in the Pfizer vaccine. The sugar helps the vaccine molecules maintain their shape during freezing.

The Moderna COVID-19 vaccine is similar. Like the Pfizer BioNTech vaccine, Moderna's vaccine also uses mRNA technology to build antibodies against COVID-19. The Moderna vaccine also uses lipids to help deliver the mRNA to the cells including SM-102, 1,2-dimyristoyl-rac-glycero3-methoxypolyethylene glycol-2000 [PEG2000-DMG], cholesterol and 1,2-distearoyl-snglycero-3-phosphocholine [DSPC]. Other ingredients include acids, acid stabilizers, salt and sugar all work together to maintain the stability of the vaccine after it's produced: acetic acid, acid stabilizers tromethamine and tromethamine hydrochloride. The vaccine also includes sodium acetate and sucrose. All of these reagents help form the vaccine nanocomplex, the structure that is injected into the body.

The recipe for these vaccines comprises the active constituent, a sequence of genetic material (DNA, RNA) that encodes the information to produce the antigen in host cells. The vaccine is administered with an adjuvant to facilitate the innate immune response. Both components are usually embedded in a carrier that protects the active ingredient and properly delivers it to the host. An adjuvant is a substance added to some vaccines to enhance the immune response of vaccinated individuals. Some examples of adjuvants in some vaccines are aluminum hydroxide, aluminum phosphate, alum (potassium aluminum sulfate), or mixed aluminum salts. The adjuvants AS03, M F59, and CpG 1018 produced by GlaxoSmithKline, Seqirus, and Dynavax are available for COVID-19 vaccine development.

The transfection reagents are lipids or related and associated materials that form a nanocomplex structure. The nanocomplex structure is the carrier having the function of delivering the key ingredient (e.g., mRNA sequence or plasmid sequence).

While these vaccines have been shown to be extremely effective. the vaccines must be stored at freezer temperatures, −18° C. to −20° C., or lower at laboratory freezer temperatures −20° C. to −80° C. or even −85° C. to prevent vaccine degradation and to preserve efficacy.

The lipid or transfection and other vaccine additives reagents may provide protection to the encapsulated nucleic acids. For example, RNase enzyme is ubiquitous and exposure to this enzyme will degrade RNA. DNase will degrade DNA. The vaccine additives can shield the nucleic acid from these destructive enzymes. Nevertheless, cold temperatures are needed to keep these Covid vaccines stable and maintain efficacy.

There is a need to design, formulate and preserve vaccines that are stable at refrigerated temperatures such −4° C. to and even warmer ambient temperatures and room temperature. Current vaccines must be kept cold for the entire process from production of the vaccine to administration of the shot. Any break of this cold chain where the vaccine is accidently warmed will cause the vaccine to be suspect. Suspect vaccines cannot be used. Thus the current vaccines are not rugged. Because of this, many parts of the world do not have access to these nucleic acid vaccines. Electricity, freezing and roads may be unreliable or even nonexistent. Temperature stable vaccines where vaccines can be stored at higher temperatures or at least may be exposed to higher temperatures while remaining stable and not reducing their efficacy would provide much needed access to the vaccines to many places around the world that current cannot be served.

### SUMMARY OF THE INVENTION

The principle of the invention is to keep the components of the vaccine, in particular the nucleic acid, separate and stable from the transfection reagents and other reagents. The reagents stored separately are stable at freezer, refrigerator and room temperature or ambient temperature conditions. Before use as a vaccine, the nucleic acid and reagents, including cationic lipids, cationic polymers, etc., are combined to assemble and form the vaccine complex. The vaccine is then ready to be administered.

In some embodiments of the invention, the formation of the vaccine complex is a self-assembly or spontaneous assembly process. In some embodiments, the nucleic acid and reagents are mixed with pumping channels to form nanoparticles to form the assembled complex. The assembled vaccine complex is stable at freezer, refrigeration or room temperature conditions however, after mixing, it is only stable for a relatively short time. Injection (or another method of administration) may be performed within minutes, hours or days depending on the extent exposure of the vaccine to degradation conditions, such as heat.

The vaccine is prepared as two or more separately stored components and used in a two-step vaccine preparation process performed at the point of use prior to administration of the vaccine. In the first step nucleic acids and other vaccine components are stabilized and stored. In some embodiments of stable storage, the nucleic acid is lyophilized, freeze dried or precipitated. In some embodiments, the nucleic acid is stored as a dry precipitate. In some embodiments, it is a solid in a liquid phase containing precipitation solvents or stabilization solvents free of RNase or DNase. In some embodiments, it is stored in a liquid phase containing nucleic acid solvents or stabilization solvents free of RNase or DNase containing other stabilization features such as low pH or the absence of light.

In some embodiments, stable storage of the nucleic acid is performed by immobilization of the nucleic acid on a solid phase surface media. The nucleic acid can be immobilized on the surface through electrostatic forces, precipitation, non-polar interactions, hydrophobic interactions, hydrogen bonding or other polar interactions. In some embodiments, the nucleic acid can be stored on a solid media surface with an interstitial liquid phase. In some embodiments, the solvent may solvate the nucleic acid. In some embodiments, the solvent may be a nucleic acid precipitating solvent. The solvent may be a stabilization solvent free of RNase or DNase. The solid surface may be media contained in a column. The column bed may be contained in a syringe. The liquid phase associated with the immobilized surface can be aqueous or water-miscible and may contain salts, ion pairing reagents, etc. The liquid phase may also contain water-miscible organic solvents. The water-miscible solvents may be protic or aprotic organic solvents.

The stored, stable RNA or DNA may be stored in a syringe, vial, or any type of container. The stored, stable RNA or DNA may be stored under vacuum, inert gas or air. The stored, stable RNA or DNA may be stored in a container or packaging that shields the contents from light.

Nucleic acids are stable at freezer temperatures, refrigerated temperatures, room temperature and ambient temperatures. Stabilization can be performed by keeping the RNA immobile, by keeping RNase and other molecules away from the nucleic acid molecule and by disabling RNase or DNase if they are present. These nucleases can be disabled through denaturation, selective inhibition or digestion. Decomposition enzymes such as nucleases may be removed from solvents or may be destroyed by heating at high temperature or by using protein digestion enzymes.

In step two, the stabilized nucleic acid is mixed with transfection and other vaccine complex reagents to prepare the vaccine nanocomplex. In some embodiments, the stabilized nucleic acid is eluted from a column or removed from a container prior to the mixing step. In some embodiments, the nucleic acid is dissolved by the transfection reagent and other vaccine reagents. The transfection reagent may be a cationic lipid or cationic polymer or molecule. Other vaccine complex forming reagents include salts, acids, carbohydrates, sugars and proteins.

The nucleic acid and reagent mixing may be performed at room temperature by any means including a vortex, flow channels, pipette, syringe, manual or other mixing procedure/device. In some embodiments of the invention the mixing is performed at temperatures suitable to form the nanocomplexes. In some embodiments, the complex is formed by self-assembly. In some embodiments, the assembly is performed with a device to agitate or mix the reagents. Once the nucleic acid complex or particle is formed, the vaccine is ready for administration.

In a typical mRNA vaccination, each patient receives 100 μg mRNA per shot. 100 μg of mRNA can be immobilized on approximately 100 μL of resin bead media. The current mRNA vaccine doses for COVID-19 range from 30 micrograms (Pfizer/BioNTech) to 100 micrograms (Moderna). In some clinical trials, lower doses of the Pfizer/BioNTech vaccine were also successful in eliciting an immune response. CureVac has developed a COVID-19 mRNA vaccine with a dose of 12 micrograms through a combination of innovations in mRNA sequence and lipid formulations.

In one example, anion exchange media beads are contained on a syringe chromatographic column where the bed of the column is located at the distal (syringe needle) end of the column and the syringe plunger is located above the bed. One example of a resin that can be contained in the column is TOSOH DEAE, a weak base anion exchanger. mRNA suitable for a vaccine is loaded onto the resin. The loading solvent may be aqueous or partially aqueous. It can contain an organic solvent such an ethanol or another solvent. The column media with the mRNA loaded is washed with RNase-free water until no trace of RNase remains in the column or the resin bed. The water wash may also contain alcohol or other reagents to disinfect the column and resin bed. The column loaded with mRNA may be stored indefinitely at refrigeration temperatures, ambient temperature or room temperature.

In some embodiments, the interstitial liquid may be removed after the nucleic acid is loaded and the nucleic acid may be storied without liquid in a type of lyophilized or solvent removed state. The column may also be stored under vacuum. In other embodiments, the mRNA may be stored in a lyophilized or dry state in the syringe with no resin, media or substrate. The syringe may contain an inert gas, air or vacuum. In other embodiments, the mRNA may be in a vial or container. The nucleic acid in the vial or container may be bound or precipitated to a substrate or may be in a dry or lyophilized state in vacuum, air or inert gas.

When the mRNA is ready to be used, the nucleic acid may be flushed from the column or vial with aqueous-based solvent. A similar procedure may be used for a DNA or plasmid vaccine. Any type of RNA may be used in the invention. In one embodiment of the invention, mRNA can be eluted from DEAE resin Tosoh, Inc.) with sodium chloride. Elution can be performed into the transfection medium, followed by mixing to form the nanocomplex micelles. In some embodiments, sodium chloride or another salt is contained in the transfection reagent mixture and can be used to elute the RNA. In one example, a 500 mM NaCl elution solvent is diluted by a factor of 3 or more resulting in approximately 150 mM sodium chloride in the final 1 mL injection. A similar procedure may be used for a DNA vaccine.

In some embodiments, a charge switch resin may be used. In some embodiments, a chaotropic silica may be used. In some embodiments, a hydrophobic interaction surface may be used.

The mRNA (or other nucleic acid) can be eluted directly into the transfection reagent mixture and the associated complex forming reagents. Mixing can be performed by any method including mixing within a syringe by moving the plunger back and forth repeatedly, pipetting up and down, vortexing, pumping channels or with any other mixing procedure or device. Formation of the vaccine complex may be performed at room temperature. The reagents can include lipids, polyamines, and other reagents such as those used for RNA, plasmid or DNA transfection reagents. This process is stable and can be performed in at room temperature.

For the purpose of this invention, this method and device can be an injection device. The nucleic acid is kept stable when it is immobilized on the solid phase or as a lyophilized or dry solid. The nucleic acid can be removed from the column or vial or contained and mixed with temperature stable micelle or transfection reagents and associated reagents, forming an enveloped RNA or enveloped DNA. The mixture can be injected into muscle. The RNA (or other nucleic acid) enters the cells and uses the cell machinery to produce the desired proteins. The produced proteins trigger an immunological response.

### BRIEF DESCRIPTION OF THE FIGURES

FIG. 1. Stabilized nucleic acid on a resin media contained in a syringe column.

FIG. 2. Stabilized precipitate, lyophilized or dissolved liquid nucleic acid contained in a syringe.

FIG. 3A. Stable nucleic acid is mixed with a nanocomplex reagents and associated reagents. FIG. 3B The nanocomplex vaccine is formed.

### DEFINITIONS

DNA (deoxyribonucleic acid) and is defined herein to include naturally occurring, chemically or enzymatically modified bases variants thereof. DNA includes single stranded, double stranded or any variation of DNA. The DNA may be a plasmid.

RNA is defined herein to include all naturally occurring, chemically or enzymatically modified bases and variants thereof. RNA includes single stranded, double stranded or any variation or structure of RNA. RNA may be messenger RNA (mRNA), ribosomal RNA (rRNA), transfer RNA (tRNA), small nuclear RNA (snRNA), regulatory RNAs, transfer-messenger RNA (tmRNA), small interfering RNA (siRNA), ribozymes (RNA enzymes), single-stranded, RNA (ssRNA), double-stranded RNA (dsRNA) and other forms.

Fusogenicity is defined as the ability to facilitate fusion as related to cells.

Lyophilization is defined to include any type of drying process with or without an additive.

Precipitated nucleic acid is defined to be any solid form of nucleic acid.

Vaccine is defined herein as a treatment or prevention of any illness, sickness, ailment or disease.

Nanocomplex reagents are defined herein as any non-nucleic acid vaccine component. The following are examples of reagents that can be included in the nanocomplex reagents described herein: transfection reagent, lipids, polymers, transfection agents, transfection medium, transfection micelle reagents, cationic liposomes, polyethylenimine-based reagents, lipid-based reagents, or cationic liposome reagents, and can include many other reagents including acids, buffers, salts, stablizers, proteins, sugars, carbohydrates, solvents, adjuvants, etc.

The following terms represent complexes that can be used in the instant invention: nanocomplex micelle, vaccine nanocomplex, nucleic acid transfection complex, transfection molecule, micelle nanocomplex, lipid nanoparticle, nucleic acid carrier envelope, transfection nanoparticle, lipid nanocomplex, nucleic acid complex, nucleic acid particle, cationic liposomes, vaccine complex, particle, micelle, cationic lipid complex, cationic polymer complexes and transfection micelle.

Cationic lipid complexes can also be called lipoplexes.

Cationic polymer complexes can also be called polyplexes.

Injection is defined to include any method of vaccine administration with any type of device including syringe, needles, pad, stream, etc. Oral administration is also included.

### DETAILED DESCRIPTION OF THE INVENTION

The principle of the invention is to keep the two parts of the vaccine separate from each other and stable. The two parts of the vaccine are 1) the nucleic acid and 2) the transfection reagents and associated reagents. These two parts are separated from each other and each component is stable from degradation. Prior to administration of the vaccine, the two parts are combined to form the vaccine complex. The two parts are stable and can be stored long term. The two parts can be stored for one week or less, one month or less, one year or less, or at least one year.

The two vaccine parts are stable at freezer, refrigeration, ambient or room temperature conditions. Room temperature or ambient temperature storage conditions are commonly used. Final preparation of the vaccine is performed right before use. After mixing and assembly, the vaccine can be administered to a person or an animal. The vaccine may be delivered in any manner including intramuscular, subcutaneous, intravenous, transdermal or oral. The vaccine may be administered within seconds, minutes, hours or days.

The vaccine of the invention is prepared in a two-part process. RNA (or any nucleic acid or combination of nucleic acids) is stored and stabilized. In some embodiments of the invention, the nucleic acid is immobilized as a solid. The nucleic acid can be stabilized and stored by immobilization as a solid on a solid phase surface using any method including precipitation, electrostatic forces, non-polar interactions, hydrophobic interactions, hydrogen bonding or other polar interactions. The nucleic acid may be stored as a solid through lyophilization, precipitation, electrostatic forces, non-polar interactions, hydrophobic interactions, hydrogen bonding or other polar interaction. The nucleic acid may be stored as a liquid or a solid.

The nucleic acid may be deposited or collected on a solid surface. The solid surface on which nucleic acid is immobilized may be media contained in a column such as a column bed. The column bed may be contained in a cartridge or column body. The column bed or solid nucleic acid may be contained in a syringe, vial, chamber or any type of container. The structural components of RNA and DNA allow for precipitation, lyophilization or several different types of solid phase media immobilization. The mechanism of immobilization may be ion exchange, ion-pairing interaction, chaotropic interaction, hydrophobic interaction or precipitation or other mechanisms. Ion exchange may include strong base ion exchange, weak base ion exchange or charge switch ion exchange

The nucleic acid may be dissolved in a liquid, precipitated, lyophilized, or bound to media or bound to media and precipitated. In some embodiments, the nucleic acid portion of the vaccine is held under vacuum. In some embodiments, the nucleic acid portion of the vaccine is shielded from light.

The nucleic acid contained in the column may be sorbed onto a surface with no interstitial fluid surrounding the media. In some embodiments, there can be an interstitial liquid surrounding the media. The interstitial liquid phase associated with the immobilized surface can be an aqueous, water-miscible organic solvent or a mixture of solvents and may contain salts, ion pairing reagents, or other reagents to prevent degradation, etc. The interstitial liquid phase surrounding the media may promote precipitation of the nucleic acid onto the media. In another embodiment of the invention, the nucleic acid may be present as a solid. The solid in the column may be lyophilized or it may be a precipitated solid containing nucleic acid. For example, ethanol may be used to precipitate the nucleic acid onto the media. In another embodiment of the invention, the nucleic acid may be in a liquid form that is kept separate from the nanocomplex reagents.

In this invention, nanocomplex reagents are defined, apart from the nucleic acid, as all of the reagents necessary to form the vaccine complex including lipids, polymers, salts, sugar, carbohydrates, proteins and pH modifying materials. However, the invention does not exclude the stable storage of one or more of these reagents with the nucleic acids. For example a salt, protein or sugar may be stored with the nucleic acid and rest of the nanocomplex components stored separately. Therefore, in defining this invention, the nucleic acid share or portion of the invention may contain additional materials and the nanocomplex reagent share or portion of the invention may be missing one or more materials. The RNA or other nucleic acids are stable at freezer and refrigerated temperatures, room temperature and ambient temperatures. Stabilization can be performed by many different methods including keeping the nucleic acids immobile, by keeping RNase and other molecules away from the nucleic acid molecule, by preventing hydrolysis or other degrading reactions, or by removing or disabling RNase or DNase if present.

Charge switch elution of mRNA is performed at neutral pH. Elution can be performed into the transfection reagents and mixed. The transfection micelle reagent can be used at a neutral pH to perform the elution. The elution action promotes mixing, and the transfection micelles are formed at room temperature. In another embodiments, the mixing is done after elution of the RNA into the transfection reagent with another syringe, vortex, etc.

Ion pairing reverse phase chemistry retains nucleic acids on a non-polar surface. In one proposed mechanism, an ion pairing reagent such as triethylammonium acetate (TEAA) is added to the nucleic acid to form a neutral ion pair that is then immobilized on the non-polar surface. The nucleic acid is removed from the surface by increasing the concentration of the water miscible organic solvent to remove the neutral ion pair. Other mechanisms have been proposed for ion pairing but, in all cases, the elution solvent is an increasing concentration of a water-miscible organic solvent.

Hydrophobic interaction chemistry is a separation technique that uses the properties of hydrophobicity of the nucleic acids. The salt in the buffer reduces the solvation of sample solutes, thus as solvation decreases, hydrophobic regions that become exposed are adsorbed by the medium. This is also called chaotropic chemistry. The nucleic acid is removed from the media by removing the salt from the aqueous solvent.

Hydrophilic interaction chemistry uses a polar stationary phase. For example, silica or a polar bonded phase can be used in conjunction with a mobile phase containing water, organic solvent and solvent additives. HIC occurs in four main stages: equilibration, sample preparation and wash, elution, and regeneration. Equilibration is normally performed with a salt solution. Nucleic acid adsorption is performed in the presence of high salt concentration. In elution, molecules are released by decreasing the salt concentration in the column. Finally, in regeneration all molecules still bound to the stationary phase are flushed from the column so it can be reused for another round of purification. There are several variations of the chemistry, but elution of the nucleic acid can normally be accomplished with water.

Precipitation of nucleic acids onto solid media may be used as an additional, secondary step to immobilize, stabilize and preserve nucleic acids. The nucleic acid is first sorbed to the media and then an organic solvent precipitates the nucleic acid as a second step. For example, nucleic acid may be bound to an ion exchanger, reverse phase material or silica material and then precipitated using a solvent such as ethanol. The nucleic acid may be retained by precipitation on the media by replacing the salts with a precipitating solvent such as ethanol or ethanol/water. The nucleic acid can be released or removed using water, buffer or another appropriate liquid.

Water and low salt concentration may be used to remove the nucleic acid from a charge switch anion exchanger and higher salt concentration may be used to remove the material from the ion exchanger.

In the next part of the process, the stabilized and preserved RNA or nucleic acid is eluted from the column and mixed with transfection reagents to prepare the vaccine nanocomplex. The mixing can be performed at room temperature with a vortex, pipette, syringe, pumping channels or any other mixing procedure/device. The complex is formed by self-assembly. Once the nucleic acid transfection complex vaccine is formed, it is ready for administration for example, by injection.

Formation of the transfection molecule or micelle nanocomplex can be performed at room temperature. Useful reagents include lipids, polyamines, and other reagents such as those used for plasm id or DNA transient transfection. Vectors include cationic liposomes and polymers which deliver biologically active agents, such as plasmid DNA, small interfering RNA (siRNA), and mRNA forming an enveloped RNA. These reagents are prepared to be as free as possible of nucleic acid degrading enzymes including RNase and DNase.

Various carriers have been used to enhance the uptake and expression of mRNA to increase the efficacy of vaccines. One commercial example is InstantFECT PGR-Solutions (Pittsburgh, PA, USA), a liposome system that forms a nanocomplex with a nucleic acid. The reagents are sterile and remain stable at refrigeration temperatures and room temperature. Many synthetic nucleic acid delivery agents are either liposomes or polymers that carry cationic charges. When combined with nucleic acids, these reagents form nucleic acids into nanoparticle complexes. RNA and DNA may be enveloped with different chemicals including linear polyethylenimine-based reagents or lipid-based reagents and many others. The complexes are sometimes called lipid nanocomplexes or cationic liposomes. Other reagents, some of them proprietary, can be used as transfection reagents.

Transfection is the process for delivering exogenous nucleic acid into cells. Transfection reagents are the most powerful alternative to viral vectors for nucleic acid delivery. They are easy to use, cost-effective and considered safe and efficient vehicles for RNA delivery. mRNA transfection is rapidly emerging as a promising method for nucleic acid-based therapy and offers an attractive substitute for plasmid DNA. Non-viral mRNA delivery methods have already proven their efficiency in vaccination through the modification of antigen presenting cells and in anti-cancer therapy by directly targeting malignant cells. The intrinsic advantage of mRNA-based immunotherapy relies on the self-adjuvant activity of mRNA and the fact that small amounts of encoded antigen are sufficient to obtain a robust immune response.

Delivery tools are equally important in the effectiveness of mRNA vaccines and therapeutics. After the mRNA elution, the next consideration is the complex formation. These complexes include oligonucleotides bound to lipids forming a lipoplex or positively charged polymers such as polyethyleneimine (PEI) forming polyplexes. For example, PolyPlus In vivo-jetRNA® is a transfection reagent specifically developed to deliver mRNA in vivo. This reagent can form an RNA complex to target multiple organs, by using systemic or local injection routes, in various animal models. mRNA delivery using in vivo-jetRNA® can be used for research vaccination purposes, anti-cancer studies, genome editing using the CRISPR/Cas9 method or protein replacement.

Other examples of transfection reagents include Agilent Stratagene Mammalian CaPO4 Transfection Reagent, Agilent Stratagene DEAE-Dextran Transfection Reagent, Promega FuGENE® HD Transfection Reagent, ThermoFisher Life Technologies Lipofectamine™ 3000 Transfection Reagent, ThermoFisher Gibco ExpiFectamine™ 293 Transfection Reagent, Agilent Stratagene Mammalian Modified CaPO4 Transfection Reagent, ThermoFisher Gibco ExpiFectamine™ CHO Transfection Reagent, MirusTranslT®-2020 Transfection Reagent, and Polyplus-Transfection® PEIpro® optimized linear polyethylenimine. These reagents are generally incubated with RNA at room temperature for 15-30 minutes to allow sufficient time for complexes to form.

Lipid nanoparticles (LNP) are used for mRNA delivery. Each lipid nanoparticle consists of four different lipids allowing the mRNA to be carried inside it and protected from degradation.

Cationic/ionizable lipids encapsulate the RNA with electrostatic interactions. These lipids are also responsible for efficient release of the RNA into the cytoplasm. The structure of a cationic lipid will affect its activity, toxicity and biodistribution and potential toxicity in the body.

Polyethylene glycol (PEG) lipids provide colloidal stability and prevent protein binding to the nanoparticle, shielding the nanoparticle and the nucleic acid from the immune system and achieving longer circulation. The length of the PEG chain and fatty acid chains determine the circulation lifetime and how well the particle can fuse with the endosomal membrane of the LNP. If the goal is prolonged circulation, longer fatty acid chains can be used, such as polyethylene glycoldistearoylglycerol (DSG PEG 2000). The concentration of PEG also influences the size of the particle. In addition, use of PEG may result in the formation of antibodies against it, potentially rendering the immunization useless.

Neutral/anionic lipids provide structural stability and play a role in defining the fusogenicity and biodistribution. Two examples are 1,2-dioleoyl-sn-glycero-3-phosphoethanolamine (DOPE) and 1,2-distearoyl-sn-glycero-3-phosphocholine (DSPC). These helper lipids may assist in the stable encapsulation of the RNA.

Cholesterol is used to modulate the bilayer density, fluidity and uptake of the LNP. There are animal-derived and synthetic versions of cholesterol available. The lipid types are chosen based on the delivery system to achieve maximum efficacy and optimal biodistribution. In addition to the choice of lipids, the ratio between the individual lipids is important to consider and fine tune, as it has a direct impact on the bilayer fluidity and the fusogenicity of the LNP.

Several critical aspects must be considered when selecting the lipid. Lipid type, source and quality have a direct impact on the impurity profile and properties such as the nanoparticle characteristics, stability and release profile in the final formulation. To achieve reproducible results with the final formulation, consistent quality of lipids is required, which is dependent on the quality of the raw materials used to synthesize the lipids and appropriate material characteristics of the lipid itself.

The purified mRNA can be formulated into the delivery particle via different techniques. In one solvent injection technique, lipids are dissolved in a solvent such as ethanol and rapidly mixed in an aqueous, low pH buffer containing the mRNA. Mixing may be performed using crossflow mixing or microfluidic mixing to create the LNPs. The low pH buffer is then diafiltered into a neutral buffer and ultrafiltration is used to concentrate the particles.

The tangential flow filtration (TFF) step must be rapid as lipids can be hydrolyzed at low pH, leading to formation of impurities such as hydrolipids that can affect the lipid bilayer structure, stability of the formulation and drug release characteristics. Degradation of the lipids can also increase the size of the particle, resulting in aggregation.

LNPs have very good stability, structural plasticity and enhanced gene delivery compared to other delivery systems. They vastly increase the transfection rate compared to transfection of naked mRNA, allow for intravenous injection without the risk of degradation by RNases present in the bloodstream. In addition, they enable active targeting if specific ligands are incorporated.

Disadvantages of LNPs include the fact that they may require cold chain logistics. In addition, sterile filtration is not always possible with LNPs and in such cases alternatives, such as gamma irradiation, heat sterilization, high-pressure sterilization or closed processing must be considered.

LNP formation by simple vortexing of ethanol lipid solutions and aqueous mRNA is possible. A microfluidic mixing device may be used to form LNPs and encapsulate mRNA. Lower lipid concentrations in ethanol may produce smaller LNP vesicles. For example, in one study, a 5 mg/mL lipid solution resulted in generally smaller LNPs than 20 mg/ml lipid solutions. Furthermore, at higher mixing flow rates, smaller LNPs are formed. For example, at a flow rate of 500 μL/min the LNPs formed were smaller on average than at 100 μL/min. Flow rates can also be adjusted in terms of a ratio of the flow rate of the aqueous solution to the flow rate of the lipid solution. At a flow rate of 3 (meaning the aqueous solution is being pumped through the orifice at 3 times the rate of the lipid solution), LNPs were larger than at a flow rate ratio of 4. To use a microfluidic mixer, one reservoir of the device can be loaded with the previously-prepared mRNA solution and the other reservoir can be loaded with equal volume of the lipid/ethanol solution.

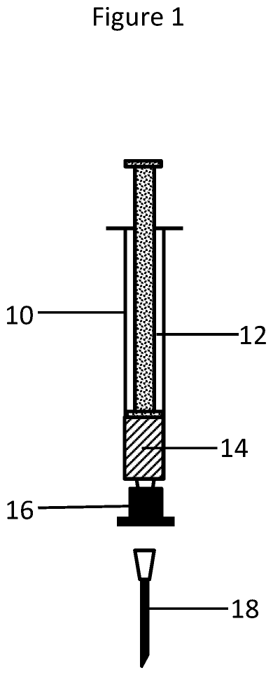

FIG. 1 shows a syringe 10 containing stabilized nucleic acid on media bed resin 14 with a syringe plunger 12 positioned above the media bed. The syringe may be capped with luer cap 16 or needle 18. In one example the resin is a DEAE anion exchanger loaded with mRNA. The mRNA is immobilized and free from RNase.

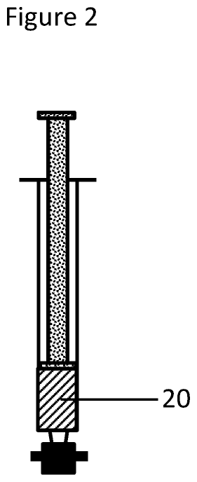

FIG. 2 shows a stabilized precipitate, lyophilized, or solution of nucleic acid 20 contained in a syringe. The nucleic acid is stable at room temperature or ambient temperature but may be also stored at lower temperatures.

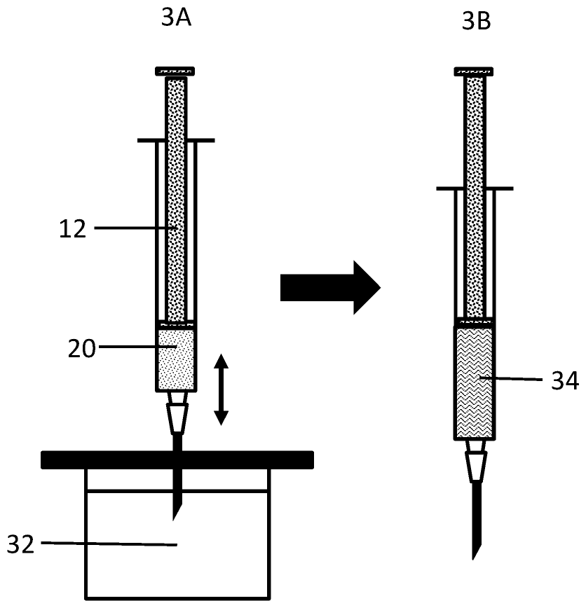

In FIG. 3A, nucleic acid 20 is mixed with nanocomplex reagents 32. Plunger 12 can be retracted to draw nanocomplex reagents 32 into the media bed. Plunger 12 can be moved up and down (back and forth) to facilitate mixing of nanocomplex reagents 32 with nucleic acid 20. In different embodiments, nucleic acid can be dispensed or first dissolved and then dispensed or can be eluted from the media bed and then mixed with a nanocomplex reagents 32. In FIG. 3B the vaccine nanocomplex 34 is formed and ready to be used for vaccination.

The vaccine nanocomplex 34 is dispensed from the syringe after mixing. N. In one example, nucleic acid 20 is bound to a DEAE anion exchange resin and the nano complex reagents are comprised of 1% NaCl which displaces the mRNA from the DEAE anion exchange sites. Following the steps shown in FIG. 3B, the nanocomplex vaccine is formed and is ready for injection.

### Example 1

To make the vaccine, elution from DEAE resin (TOSOH BIOSCIENCE) is performed with approximately 300 μl of 0.5 M sodium chloride resulting in an approximate concentration of 150 mM sodium chloride in the final 1 mL injection. Elution is performed directly into the transfection medium, and then mixed to form the micelle complex for injection. mRNA is retained and stored on a syringe column containing 3 mL of DEAE resin. The resin surface is fully loaded with mRNA AdsA. AdsA encoded a therapeutic protein to combat S. aureus skin infection disease. RNase has been eluted and removed from the anion exchanger. The mRNA is eluted with 10 mL of 500 mM NaCl. 10 mg of AdsA mRNA is diluted with 10 mL of serum-free medium. Then, 20 mL of 0.9% NaCl is added, and 4 mL of InstantFECT is added into the solution. All reagents are sterile and nuclease free. The final mixture is pipetted several times and injected into mice.

Capture immobilization on a charge switch ion exchange resin of the mRNA is performed under slightly acidic conditions, e.g., pH 5. The mRNA is then washed and stabilized in 70% ethanol, perhaps 95% ethanol. The interstitial liquid is removed and the mRNA is “syringe stable” at room temperature indefinitely. Acetonitrile or other aprotic or protic water miscible solvent could be used, but the resin would have to be substantially cleaned from this solvent before elution. Water or dilute buffer or the transfection reagent at neutral pH can be used to elute the nucleic acid from the anion exchanger. The nucleic acid is mixed with the transfection reagent to form the vaccine.

Nucleic acid can be adsorbed and stored on silica. Nucleic acid can be added to silica under chaotropic conditions. The nucleic acid is adsorbed to the surface and then the silica is washed with 90% ethanol. mRNA or other nucleic acids which can be stored under these conditions. Optionally, the interstitial alcohol can be removed by blowing out excess liquid or even (partially) drying the column, leaving behind the stable mRNA. When ready to form the vaccine, the nucleic acid may be eluted with water or dilute buffer.

An injection is made of the mixture into the muscle. RNA enters the cell and uses the cell machinery to produce the desired proteins. The produced proteins trigger an immunological response. The person is now safe from viral infection. Or the immunological response is directed toward cancer cells or other diseases. The two-part vaccine is stable at room temperature. The mRNA is stable as it is immobilized on the solid phase. The transfection reagents can be made stable at room temperature with proper sterilization. 4° C. refrigeration or freezing may be used as well.

1. A method for making a nucleic acid vaccine, comprising the steps of:
a. providing a nucleic acid, wherein the nucleic acid is stable, and wherein the nucleic acid is contained within a syringe, vial, column or chamber;
b. providing nanocomplex reagents;
c. mixing the nucleic acid with the nanocomplex reagents to produce nanoparticles, wherein the nanoparticles comprise a vaccine; and
d. administering the vaccine.

2. The method of claim 1, wherein the nucleic acid is immobilized.

3. The method of claim 1, wherein the nucleic acid is immobilized on a bed of media.

4. (canceled)

5. The method of claim 1, wherein the nucleic acid is lyophilized or precipitated.

6. The method of claim 1, wherein the nucleic acid is stored under vacuum or wherein the nucleic acid is shielded from light.

7. (canceled)

8. The method of claim 1, wherein step (c) is performed using a vortex, a pipette, pumping channels, flow channels, a syringe or manually.

9. The method of claim 1, wherein multiple doses of the vaccine are produced in step (c) and the vaccine is administered into multiple people in step (d).

10. The method of claim 1, wherein the method is performed at ambient temperature.

11. The method of claim 1, wherein the nucleic acid is immobilized on a bed of media and wherein the nucleic acid is eluted prior to performing step (c).

12. The method of claim 1, wherein the method is performed in a point of care setting.

13. The method of claim 1, wherein the nucleic acid is RNA.

14. The method of claim 1, wherein the nucleic acid is DNA.

15. The method of claim 1, wherein prior to step (b), the stabile nucleic acid and the nanocomplex reagents are stored for 1 month or less.

16. The method of claim 1, wherein prior to step (b), the stable nucleic acid and the nanocomplex reagents are stored for 1 year or less.

17-19. (canceled)

20. The method of claim 1, wherein the nucleic acid is contained within a syringe, wherein step (b) is performed by drawing the nanocomplex reagents into the syringe through the distal end of the syringe, wherein step (c) is performed within the syringe and wherein step (d) is performed by injection with the syringe.

21. The method of claim 1, wherein the nanoparticle is a nucleic acid delivery particle.

22. The method of claim 1, wherein the nucleic acid is dissolved by a transfection reagent prior to step (c).

23. The method of claim 22, wherein the transfection reagent is lipid based.

24. The method of claim 1, wherein the nucleic acid and the nanocomplex reagents are stored for at least 1 year.
